(19) United States
(12) Patent Application Publication
(10) Pub. No.: US 2023/0086091 A1
(43) Pub. Date: Mar. 23, 2023

(54) APTAMERIC PEPTIDE LIBRARY FORMATION USING GENERATIVE ADVERSARIAL NETWORK (GAN) MACHINE LEARNING MODELS

(71) Applicant: University of Florida Research Foundation, Incorporated, Gainesville, FL (US)

(72) Inventors: Zachary F. Greenberg, Gainesville, FL (US); Mei He, Gainesville, FL (US); Kiley S. Graim, Gainesville, FL (US)

(21) Appl. No.: 17/903,287

(22) Filed: Sep. 6, 2022

Related U.S. Application Data
(60) Provisional application No. 63/242,848, filed on Sep. 10, 2021.

(51) Int. Cl. C07K 1/04; G06N 20/00; G06N 3/08

(52) U.S. Cl. CPC C07K 1/047; G06N 20/00; G06N 3/08

(57) ABSTRACT

Various embodiments generally relate to intelligently designing aptameric peptides for binding with a specific receptor and forming aptameric peptide libraries with the designed peptides. The aptameric peptides libraries can be tissue-specific and be used in drug delivery and therapeutic applications, in which designed peptides can be implanted on exosome surfaces for exosomal cargo delivery to a specific tissue. Various embodiments of the present disclosure involve the use of a generative adversarial network (GAN) machine learning model configured (e.g., trained) and used to output designed peptides that are similar to pre-existing peptides of a peptide dataset but that specifically bind to a selected receptor and have various selected physiochemical properties. In various embodiments, GAN machine learning models may receive representations of the pre-existing peptides and may output representations of designed peptides according to peptide vectorization and encoding schemas based at least in part on the amino acids within a peptide.

### REFERENCE TO RELATED APPLICATIONS

This application claims priority to and the benefit of U.S. Provisional Application. No. 63/242,848, filed on Sep. 10, 2021, the entire contents of which are incorporated herein by reference.

### ACKNOWLEDGEMENT OF GOVERNMENT SUPPORT

This invention was made with government support under R35 GM133794 awarded by the National Institutes of Health. The government has certain rights in the invention.

### TECHNOLOGICAL FIELD

Various embodiments relate to intelligent generation of aptameric peptide libraries for specifically selected target receptors. Some example embodiments may further relate to and/or be applied to drug delivery and therapeutics when implanting aptameric peptides from the intelligently generated aptameric peptide libraries onto exosome surfaces for exosomal targeting, trafficking, signaling, and/or homing.

### BACKGROUND

Exosomes have been emerging in developing drug delivery and therapeutics, due at least in part to their natural biocompatibility, fast cellular uptake, and receptor mediated specific tissue targeting. Exosomes, which are typically have diameters between approximately 30 nanometers (nm) and approximately 50 nm, are derived from endosomal origin and carry enriched collections of membrane proteins, cytokines, enzymes, certain lipid rafts, as well as RNAs (e.g., miRNAs, non-coding RNAs, tRNAs, rRNAs) and DNAs, each of which are essential signaling components and are uniquely tied to the cellular origin of an exosome. Exosomes have an intrinsic ability to mediate cellular communications via targeting different cellular phenotypes, which can be exploited in pharmaceutical drug delivery systems. Various embodiments of the present disclosure address technical challenges related to precise use of exosomes in tissue targeting and relevant immunity activation.

### BRIEF SUMMARY

Various embodiments provide methods, systems, apparatuses, computer program products, and/or the like for generating aptameric peptide libraries using a generative adversarial network (GAN) machine learning model. The aptameric peptide libraries comprise, describe, identify, and/or the like peptides that are configured to uniquely bind with a specific cellular receptor (e.g., tissue-specific receptors), and these peptides can be attached to the surface of exosomes, such that the exosomes can target the specific cellular receptor, for example. In one example embodiment, a GAN machine learning model is used to generate an aptameric peptide library specifically targeting major histocompatibility complex presentations (e.g., MHC-I receptors, MHC-II receptors).

In various embodiments, a GAN machine learning model is configured (e.g., trained) to generate an aptameric peptide library comprising a plurality of peptides using a peptide dataset having pre-existing peptides and comprises a generator and a discriminator. The generator of the GAN machine learning model is configured to output designed peptides that are similar to the pre-existing peptides, and the designed peptides may be completely synthetic and functional without having previously existed. Meanwhile, the discriminator is configured to generally classify the binding ability (e.g., a binary classification of binding or non-binding) of a peptide with a target receptor and can be used to classify the binding ability of designed peptides output by the generator. The discriminator may be configured to classify binding ability based at least in part on selected physiochemical properties. Within the GAN machine learning model, designed peptides are generated and classified in an iterative manner until a plurality of designed peptides with optimized binding with a target receptor is determined and can be used to form the aptameric peptide library. The designed peptides output by the GAN machine learning model (or specifically the generator thereof) may be referred interchangeably herein as generated peptides, targeting peptides, artificial peptides, engineered peptides, synthetic, latent peptides, evolved peptides, derived peptides, and/or the like.

In accordance with one aspect, a method is provided. In one embodiment, the method comprises receiving a selection of a target receptor; encode a plurality of pre-existing peptides from a peptide dataset; configuring a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and generating an aptameric peptide library comprising a plurality of designed peptides output by the generator of the GAN machine learning model, the aptameric peptide library being specific to the target receptor.

In accordance with yet another aspect, an apparatus comprising at least one processor and at least one memory including computer program code is provided. In one embodiment, the at least one memory and the computer program code may be configured to, with the processor, cause the apparatus to receive a selection of a target receptor; encode a plurality of pre-existing peptides from a peptide dataset; configure a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and generate an aptameric peptide library comprising a plurality of designed peptides output by the generator of the GAN machine learning model, the aptameric peptide library being specific to the target receptor.

In accordance with another aspect, a computer program product is provided. The computer program product may comprise at least one computer-readable storage medium having computer-readable program code portions stored therein, the computer-readable program code portions comprising executable portions configured to receive a selection of a target receptor; encode a plurality of pre-existing peptides from a peptide dataset; configure a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and generate an aptameric peptide library comprising a plurality of designed peptides output by the generator of the GAN machine learning model, the aptameric peptide library being specific to the target receptor.

### BRIEF DESCRIPTION OF THE SEVERAL VIEWS OF THE DRAWING(S)

Having thus described embodiments of the present disclosure in general terms, reference will now be made to the accompanying drawings, which are not necessarily drawn to scale.

FIG. 1 is an overview of an example system architecture for generating tissue-specific aptameric peptide libraries using GAN machine learning models, in accordance with embodiments of the present disclosure.

FIG. 2 is a block diagram of an exemplary apparatus that may perform various operations for generating tissue-specific aptameric peptide libraries using GAN machine learning models, according to one embodiment.

FIG. 3 is an exemplary diagram illustrating generating tissue-specific aptameric peptide using GAN machine learning models for exosomal cargo delivery, in accordance with an example embodiment.

FIG. 4A illustrates an exemplary flowchart of an example process including various operations for generating tissue-specific aptameric peptide libraries using GAN machine learning models, according to one embodiment.

FIG. 4B provides an exemplary diagram illustrating various operations for generating tissue-specific aptameric peptides using GAN machine learning models for exosomal cargo delivery, in accordance with an example embodiment.

FIG. 5A provides an exemplary diagram illustrating vectorization and encoding of peptides for a GAN machine learning model, according to one embodiment.

FIG. 5B illustrates example data describing classification accuracy of a discriminator of a GAN machine learning model in classifying binding ability of peptides encoded using various encoding schemes, according to one embodiment.

FIGS. 6A-C illustrate example data describing classification accuracy of a discriminator of a GAN machine learning model in classifying binding ability of peptides encoded using various encoding schemes, according to one embodiment.

FIG. 7A-B illustrate example data describing clustering of peptides with respect to various physiochemical features for discriminating between peptides binding with a target receptor and peptides non-binding with the target receptor, according to one embodiment.

FIG. 8 illustrates an example selected generative domain or generative space within which a generator of a GAN machine learning model is configured to generate designed peptides, in accordance with various embodiments.

FIG. 9 illustrates example data describing selection of designed peptides satisfying convergence thresholds to generate an aptameric peptide library, according to various embodiments.

FIG. 10 provides example visualizations or illustrations of peptide structures for designed peptides and pre-existing peptides, according to one embodiment.

FIG. 11 illustrates example data describing iterative performance of GAN machine learning models when outputting designed peptides that accurately bind with a target receptor and that are similar within a feature space with pre-existing peptides, according to one embodiment.

FIG. 12 illustrates example data describing benchmarking GAN's discriminatory capability on peptides against conventional machine learning approaches incorporating encoding mechanisms and feature selection, according to one embodiment.

FIG. 13 illustrates example data assessing the encoding mechanism relative to all the computational techniques, according to one embodiment.

FIG. 14 illustrates an example principal component analysis on how artificial intelligence really understands the molecular factors of antigen presentation by MHC-I, according to one embodiment.

FIG. 15 illustrates an example individual component analysis on how artificial intelligence understands the molecular factors of antigen presentation by MHC-I, according to one embodiment.

FIG. 16 illustrates an example t-distributed stochastic neighbor embedding visualization plot (t-SNE), according to one embodiment.

FIG. 17A illustrates example visualizations molecular docking of a generated peptide relative to a parent peptide, according to one embodiment.

FIG. 17B illustrates example data describing the binding strength, according to one embodiment.

FIG. 18 illustrates example data describing resulting cell viability assay by trypan blue staining determining how cells react to stimulation, according to one embodiment.

FIG. 19 illustrates example data describing the experimental binding affinity evaluation by biolayer interferometry for three pairs of peptides, according to one embodiment.

FIG. 20A is an exemplary diagram illustrating a preliminary translation of exosome vaccines by the established method using generated peptides, in accordance with an example embodiment.

FIG. 20B illustrates example data describing the concentration of a peptide with respect to the size of the peptide, according to one embodiment.

FIG. 21 illustrates example computational quality control to evaluate how the generated synthetics are molecularly similar to the naturally existing cancer antigens, according to one embodiment.

FIG. 22A illustrates the generation of synthetics in boxed regions, according to one embodiment.

FIG. 22B illustrates example data evaluating the importance of residues and the corresponding positions within MHC-I, according to one embodiment.

FIG. 23A illustrates example data describing experimental validation presumed biolayer interferometric binding strength, according to one embodiment.

FIG. 23B illustrates example data describing experimental validation presumed kinetics for generated synthetic (daughter) and the corresponding antigen counterpart (parent), according to one embodiment.

FIG. 24A illustrates an exemplary methodology workflow for an experimental validation of immunogenic activity, in accordance with various embodiments of the present disclosure.

FIG. 24B illustrates example data describing a GAN generated peptide produced immunogenic activity in four independent experiments, according to one embodiment.

FIG. 25 illustrates example data describing size distributions detailing MHC-I+enriched EVs developed from an end-to-end method, according to one embodiment.

FIG. 26 illustrates example data evaluating the performance of GAN's education and manufacturing of new MHC-I binding peptides, according to one embodiment.

FIG. 27 is a table illustrating example and selected designed peptides, in accordance with embodiments herein.

### DETAILED DESCRIPTION

Various embodiments of the present disclosure now will be described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. The terms “approximately” and “substantially” are used herein to refer to being within appropriate manufacturing and/or engineering tolerances. Like numbers refer to like elements throughout.

### I. General Overview and Exemplary Technical Advantages

Various embodiments generally relate to intelligently designing aptameric peptides for binding with a specific receptor (e.g., associated with a target tissue) and forming aptameric peptide libraries with the designed peptides. The aptameric peptides libraries may be tissue-specific and can be further used in drug delivery and therapeutic applications, in which designed peptides can be attached or implanted to exosome surfaces for exosomal cargo delivery to a specific tissue. Various embodiments of the present disclosure describe the intelligent design of aptameric peptides and formation of tissue-specific aptameric peptide libraries using generative adversarial network (GAN) machine learning models.

A GAN machine learning model is generally configured to generate new data similar to and ideally indistinguishable from a training dataset and are generally comprised of a generator and a discriminator. In various embodiments, the generator of the GAN machine learning model is configured (e.g., trained) and used to output designed peptides that are similar to peptides of a peptide dataset (e.g., existing peptides, pre-existing peptides, parent peptides, progenitor peptides, and/or similar terms used interchangeably herein). Meanwhile, the discriminator is configured to classify a binary binding ability of a peptide with a target receptor and can be used to classify a designed peptide output by the generator as a binder or a non-binder with a target receptor. Thus, in various embodiments, the GAN machine learning model is used iteratively to generate a plurality of designed peptides that bind with a target receptor. The GAN machine learning model uses native features derived from the physiochemical dynamics of receptor-epitope interaction inherently present in the pre-existing peptides of the peptide dataset to intelligently design feasible and functional aptameric peptides. In various embodiments, the GAN machine learning model may receive representations of the pre-existing peptides and may output representations of designed peptides according to peptide vectorization and encoding schemas based at least in part on the amino acids within a peptide (e.g., a pre-existing peptide, a designed peptide).

Thus, various embodiments provide various technical advantages in generating tissue-specific aptameric peptide libraries using GAN machine learning models and likewise solve various technical challenges related to efficiency of epitope-receptor selection. Existing techniques and systems for epitope-receptor selection are restricted to bench synthetic methods, which are both labor-intensive and time-intensive. In contrast, various embodiments involve intelligent and computational-based design of aptameric peptides, or epitopes, which can form aptameric peptide libraries within a short timeframe and further are more precise in targeting selected tissues and/or receptors. Computational-based design of aptameric peptides using GAN machine learning models conserves real-world resources that would otherwise be used in existing techniques for epitope-receptor selection, such as systematic evolution of ligands by exponential enrichment (SELEX) and phage displays.

Thus, precise and computational-based design of aptameric peptides, or epitopes, using GAN machine learning models and formation of tissue-specific aptameric peptide libraries enables various improvements in applications of drug delivery and therapeutics. As discussed, aptameric peptides can be intelligently designed and subsequently synthesized for implantation and attachment with exosomes, such that exosomes implanted with designed peptides (e.g., augmented exosomes) can target a specific tissue and/or receptor. Aforementioned technical advantages including the improvement to efficiency in selecting a peptide that bind with a specific receptor enable various embodiments of the present disclosure to be easily and efficiently integrated into platforms and pipelines for drug delivery, therapeutics, and/or the like. That is, various embodiments for intelligently designing aptameric peptides are advantageously scalable and also reduces variation risk across different targeting selections.

FIG. 12 illustrates example data describing benchmarking GAN's discriminatory capability on peptides against conventional machine learning approaches incorporating encoding mechanisms and feature selection, according to one embodiment. Specifically, FIG. 12 illustrates benchmarking GAN's discriminatory capability (SNN_F) on HLA*02:01 peptides against conventional machine learning approaches (SVM, NB, EN, RF) incorporating encoding mechanisms (colors) and feature selection (F=features, BA=binding affinity by itself). As shown in FIG. 12, GAN's discriminatory capability is maximized with the included features, while remaining user-friendly and reducing data loss with alphanumeric encoding. Here, SNN is an abbreviation for “shallow neural network,” SVM is an abbreviation for “support vector machine,” NB is an abbreviation for “naïve bayes,” EN is an abbreviation for “elastic net,” and RF is an abbreviation for “random forest.”

### II. Exemplary System Architecture

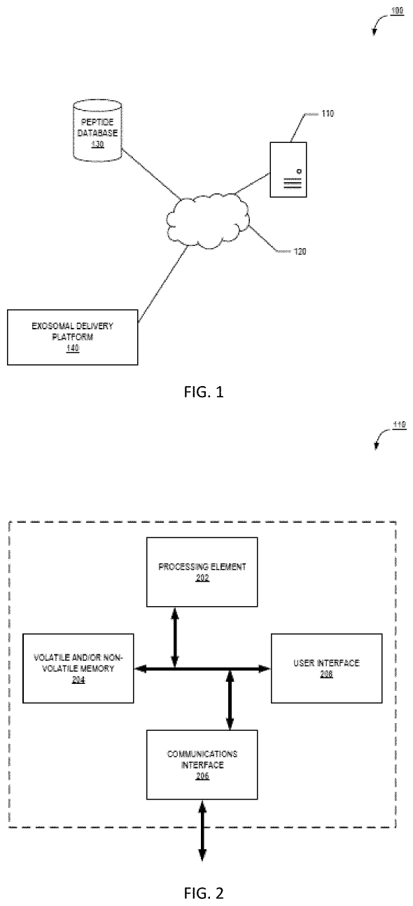

FIG. 1 provides an illustration of an exemplary system architecture 100 according to an example embodiment of the present disclosure. As shown in FIG. 1, the system architecture 100 may include one or more system computing entities 110, one or more networks 120, one or more peptide databases 130, an exosomal delivery platform 140, and/or the like. Each of these components, entities, devices, systems, and similar words used herein interchangeably may be in direct or indirect communication with, for example, one another over the same or different wired or wireless networks 120. In an example embodiment, a system computing entity 110 is in direct wired or wireless communication with a peptide database 130. While FIG. 1 illustrates the various system entities as separate, standalone entities, the various embodiments are not limited to this particular architecture. In various embodiments, the system computing entity 110 and the exosomal delivery platform 140 may be integrated into a single device or a single system.

In an example embodiment, the system computing entity 110 may be configured to communicate with a peptide database 130 that stores one or more peptide datasets. For example, the system computing entity 110 may communicate with the peptide database 130 to retrieve, receive, access, and/or the like data related to pre-existing peptides to configure (e.g., train) GAN machine learning models to intelligently design aptameric peptides (e.g., to output designed peptides). In some example embodiments, the system computing entity 110 may generate and/or update a peptide database 130 with designed peptides. That is, aptameric peptide libraries generated by the system computing entity 110 in accordance with various embodiments of the present disclosure may be stored (e.g., described by data stored) in a peptide database 130.

In an example embodiment, the system computing entity 110 may be configured to communicate with the exosomal delivery platform 140. For example, the system computing entity 110 generates a designed peptide for binding with a specific receptor (e.g., MHC-1) using GAN machine learning models and communicates the designed peptide (e.g., amino acid sequences thereof) to the exosomal delivery platform 140 for synthesis of the designed peptide and use of the designed peptide in exosomal delivery or targeting.

In this regard, in some embodiments, the exosomal delivery platform 140 is configured for customized peptide synthesis. For example, the exosomal delivery platform 140 receives an amino acid sequence, such as a sequence of a designed peptide generated by the system computing entity 110, and synthesizes peptides according to the received sequence. Various solution-phase and/or solid-phase synthesis techniques may be used by the exosomal delivery platform 140 to synthesize peptides. In various embodiments, the exosomal delivery platform 140 synthesizes an aptameric peptide library for evolution and filtering of different peptides of the aptameric peptide library.

The exosomal delivery platform 140 may be further configured for implantation and/or attachment of synthesized peptides to surfaces of exosomes. Accordingly, in some embodiments, the exosomal delivery platform 140 has access to and/or extracts exosomes from biological material for the implantation of synthesized peptides. The exosomes may be generated and extracted from biological material including cell cultures, blood, urine, milk, bacterial fluids, plant fluids, ascites, and/or the like. The exosomal delivery platform 140 may then perform and/or enable various reactions or operations to implant and/or attach synthesized peptides to exosomal surfaces. For example, synthesized peptides may be attached to exosomal surfaces via conjugation. Thus, the exosomal delivery platform 140 is configured to generate exosomes with synthesized designed peptides attached to the surfaces thereof, or augmented exosomes.

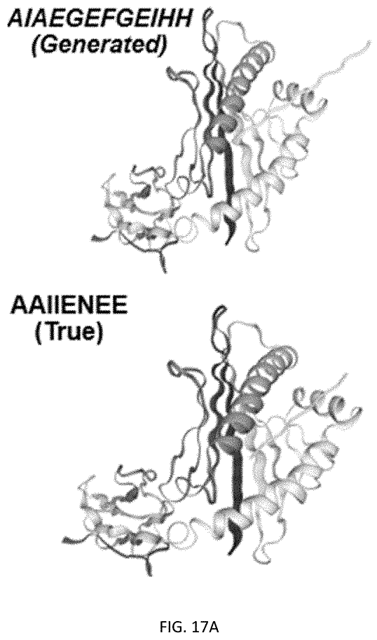

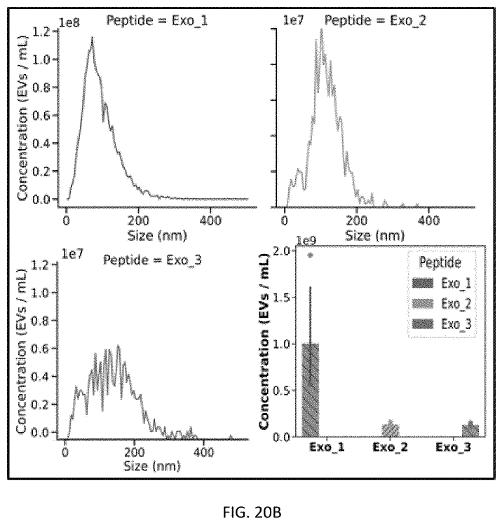

In various embodiments, the exosomal delivery platform 140 may be further configured for validation and testing of binding of designed peptides with a targeted receptor. As discussed, designed peptides are generated to bind with a specific receptor, and in some embodiments, the exosomal delivery platform 140 may screen and/or filter synthesized designed peptides based at least in part on binding capabilities with a targeted receptor. In various embodiments, the exosomal delivery platform 140 comprises various experimental assays, such as enzyme-linked immunosorbent assay (ELISA), for validating binding activity of designed peptides with the targeted receptor. Additionally or alternatively, the system computing entity 110 is configured to use informatic protein docks (e.g., the HDOCK server for integrated protein-protein docking), simulations, and/or the like to validate and test binding activity of designed peptides with the targeted receptor. FIG. 17A illustrates example visualizations molecular docking of a generated peptide relative to a parent peptide, according to one embodiment. FIG. 17B illustrates example data describing the binding strength, according to one embodiment. More specifically, as shown in FIG. 20B, the binding strength is computationally determined as how closely (tightly) the peptide is able to bind into the MHC-I receptor, according to one embodiment. A higher negative number represents a stronger affinity.

FIG. 2 provides a block diagram of the system computing entity 110, according to one embodiment of the present disclosure. The system computing entity 110 is configured to generate aptameric peptide libraries by intelligently designing aptameric peptides using GAN machine learning models. In particular, the system computing entity 110 is configured to generate designed peptides that bind with a specific receptor and may do so based at least in part on receiving a user input indicating the specific receptor. The system computing entity 110 may provide data describing a designed peptide, such as an amino acid sequence, a conformational structure, and/or the like, to the user in response to the user input, in some embodiments. In various embodiments, use of a GAN machine learning model by the system computing entity 110 to intelligently design aptameric peptides may be preempted by the system computing entity 110 configuring and training the GAN machine learning model, and the system computing entity 110 may configure and train the GAN machine learning model based at least in part on communication with the peptide database 130. In one embodiment, the system computing entity 110 may include one or more communications interfaces 206 for communicating with various other computing entities (e.g., including peptide database 130 and the exosomal delivery platform 140), such as by communicating data, content, information, and/or similar terms used herein interchangeably that can be transmitted, received, operated on, processed, displayed, stored, and/or the like.

In general, the terms computing entity, computer, entity, device, system, and/or similar words used herein interchangeably may refer to, for example, one or more computers, computing entities, desktops, mobile phones, tablets, phablets, notebooks, laptops, distributed systems, input terminals, servers or server networks, blades, gateways, switches, processing elements, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As shown in FIG. 2, in one embodiment, the system computing entity 110 may include or be in communication with one or more processing elements 202 (also referred to as processors, processing circuitry, and/or similar terms used herein interchangeably) that communicate with other elements within the system computing entity 110 via a bus, for example. As will be understood, the processing element 202 may be embodied in a number of different ways. For example, the processing element 202 may be embodied as one or more complex programmable logic devices (CPLDs), microprocessors, multi-core processors, co-processing entities, application-specific instruction-set processors (ASIPs), microcontrollers, and/or controllers. Further, the processing element 202 may be embodied as one or more other processing elements or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 202 may be configured for a particular use or configured to execute instructions stored in volatile or non-volatile media or otherwise accessible to the processing element 202. As such, whether configured by hardware or computer program products, or by a combination thereof, the processing element 202 may be capable of performing steps or operations according to embodiments of the present disclosure when configured accordingly.

In one embodiment, the system computing entity 110 may further include or be in communication with memory 204. In an example embodiment, the memory 204 comprises non-volatile media (also referred to as non-volatile storage, memory, memory storage, memory circuitry and/or similar terms used herein interchangeably). In one embodiment, the non-volatile storage or memory may include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store datasets, dataset instances, dataset management systems, data, applications, programs, program modules, scripts, source code, object code, byte code, compiled code, interpreted code, machine code, executable instructions, and/or the like. The term dataset, dataset instance, dataset management system, and/or similar terms used herein interchangeably may refer to a collection of records or data that is stored in a computer-readable storage medium using one or more dataset models, such as a hierarchical dataset model, network model, relational model, entity—relationship model, object model, document model, semantic model, graph model, and/or the like.

In one embodiment, the memory 204 may further include or be in communication with volatile media (also referred to as volatile storage, memory, memory storage, memory circuitry and/or similar terms used herein interchangeably). In one embodiment, the volatile storage or memory may also include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the datasets, dataset instances, dataset management systems, data, applications, programs, program modules, scripts, source code, object code, byte code, compiled code, interpreted code, machine code, executable instructions, and/or the like being executed by, for example, the processing element 202. Thus, the datasets, dataset instances, dataset management systems, data, applications, programs, program modules, scripts, source code, object code, byte code, compiled code, interpreted code, machine code, executable instructions, and/or the like may be used to control certain aspects of the operation of the system computing entity 110 with the assistance of the processing element 202 and operating system.

As indicated, in one embodiment, the system computing entity 110 may also include one or more communications interfaces 206 for communicating with various other computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the system computing entity 110 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The system computing entity 110 may also comprise a user interface 208 (that can include a display coupled to a processing element). For example, the user interface 208 may include or be in communication with one or more input elements, such as a keyboard input, a mouse input, a touch screen/display input, motion input, movement input, audio input, pointing device input, joystick input, keypad input, and/or the like. The system computing entity 110 may also include or be in communication with one or more output elements (not shown), such as audio output, video output, screen/display output, motion output, movement output, and/or the like. These input and output elements may include software components, such as a user application, browser, graphical user interface, and/or the like to facilitate interactions with and/or cause display of information/data from the system computing entity 110, as described herein. The user input interface can comprise any of a number of devices or interfaces allowing the system computing entity 110 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the system computing entity 110 and may include a full set of alphabetic keys or set of keys that may be activated to provide a full set of alphanumeric keys.

For example, a user of the system computing entity 110 may identify, select, and/or indicate a particular tissue and/or receptor for which one or more designed peptides and/or an aptameric peptide library is generated via user interface 208. Similarly, the user interface 208 may be provided to display various data for a designed peptide, such as amino acid sequences, conformational structures, binding affinities, various physiochemical properties, and/or the like. In some embodiments, the user of the system computing entity 110 may configure data and parameters for GAN machine learning models for the generation of designed peptides via user interface 208. User interface 208 may indicate various performance data and metrics of a GAN machine learning model, such as cross-entropy loss, simulated or computed accuracy, convergence values, iteration and/or epoch number, and/or the like.

As will be appreciated, one or more of the components of the system computing entity 110 may be located remotely from other components of the system computing entity 110, such as in a distributed system. Furthermore, one or more of these components may be combined with additional components to perform various functions described herein, and these additional components may also be included in the system computing entity 110. Thus, the system computing entity 110 can be adapted to accommodate a variety of needs and circumstances. As will be recognized, these architectures and descriptions are provided for exemplary purposes only and are not limiting to the various embodiments.

### III. Exemplary Operations

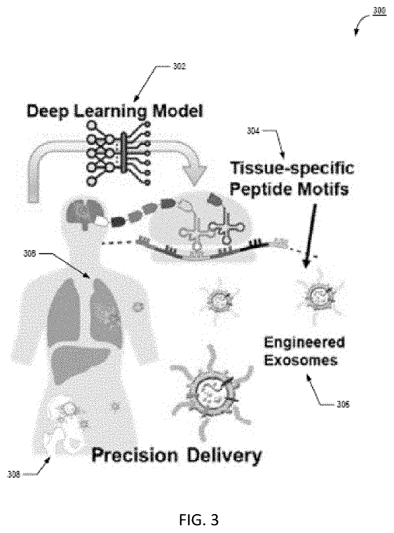

Example embodiments of the present disclosure provide for operations for intelligently designing aptameric peptides and generating aptameric peptide libraries using GAN machine learning models configured and trained using peptide datasets. In various embodiments, aptameric peptides and libraries thereof are designed to bind to a specific receptor that may be specific to a particular tissue, and aptameric peptides are implanted on exosomal surfaces to form augmented exosomes that target the particular tissue. FIG. 3 provides a diagram 300 illustrating an exemplary overview of various operations in accordance with various embodiments of the present disclosure. Diagram 300 first illustrates a deep learning model, or a GAN machine learning model 302 that outputs tissue-specific peptide motifs, or designed peptides 304. The GAN machine learning model 302 generates the designed peptides 304 based at least in part on target tissues 308 (e.g., lungs, pelvic bone) and cellular receptors thereof. Diagram 300 further illustrates synthesis of the designed peptides 304 and formation of engineered or augmented exosomes 306 that have the designed peptides 304 implanted on their surfaces. Through surface implantation and presentation of the designed peptides 304 that bind to cellular receptors of a target tissue 308, cargo (e.g., drug) delivery, homing, signaling, and/or the like to the target tissue 308 using augmented exosomes 306 is enabled.

Various embodiments provide various technical advantages in generating tissue-specific aptameric peptide libraries using GAN machine learning models and likewise solve various technical challenges related to efficiency of epitope-receptor selection. Various embodiments involve intelligent and computational-based design of aptameric peptides, or epitopes, which can form aptameric peptide libraries within a short timeframe and can further be synthesized for drug delivery and therapeutic applications. Computational-based design of aptameric peptides is more precise in targeting selected tissues and/or receptors and reduces the possible of error introduction when compared to bench synthetic techniques such as SELEX and phage display. Computational-based design of aptameric peptides using GAN machine learning models further advantageously conserves real-world resources in designing aptameric peptides for a specific receptor. Further still, various embodiments for intelligently designing aptameric peptides are advantageously scalable and reduce variation risk across different targeting selections, thereby enabling integration in industrial and manufacturing pipelines.

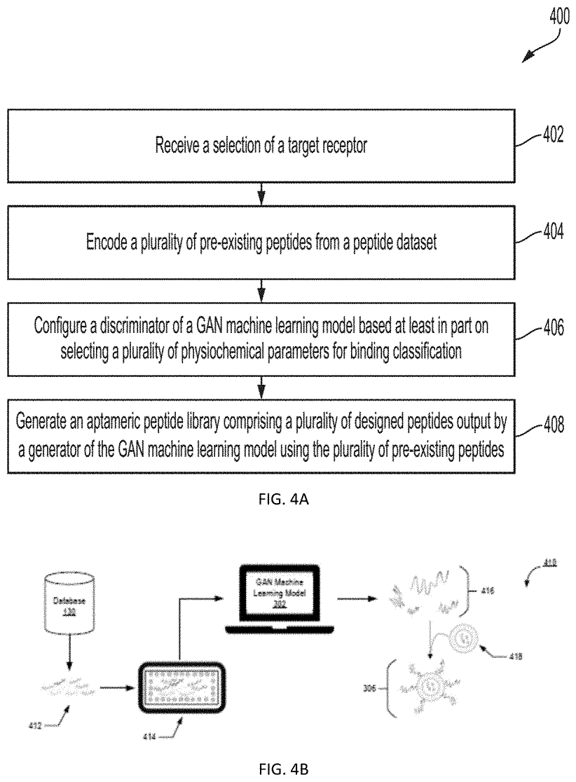

Referring now to FIG. 4A, a flowchart of an example process 400 for generating an aptameric peptide library using a GAN machine learning model is provided. Process 400 includes example steps/operations that may be performed by system computing entity 110, and the system computing entity 110 includes means, such as processing element 202, memory 204, communications interface 206, and/or the like, for performing and/or controlling each step/operation of process 400.

At step/operation 402, a selection of a target receptor is received. For example, the system computing entity 110 receives a selection of a target receptor originating from a user through user interface 208. As another example, the system computing entity 110 receives a selection of a target receptor originating from another computing entity via communications interface 206. In various embodiment, the selection of a target receptor is received as part of an application programming interface (API) request, query, call, and/or the like configured to cause a designed peptide and/or an aptameric peptide library to be generated and provided in a corresponding API response. In various embodiments, a particular tissue is provided, and a target receptor is experimentally determined, such as by using sequencing techniques.

In various embodiments, the target receptor is a cellular surface presentation or surface feature, and the selection of a target receptor represents a request to generate one or more designed peptides that bind to the target receptor. It may be appreciated that the target receptor is a receptor for protein-protein binding and may be configured to cause particular actions responsive to protein binding, such as various signaling pathways (e.g., signal transduction), metabolic pathways, and/or the like. In one example embodiment, the target receptor is the MHC-I receptor. In other example embodiments, the target receptor is a non-ubiquitous receptor specific to a particular tissue. In various embodiments, the target receptor includes multiple binding sites, and a particular binding site of the target receptor is additionally indicated in the selection of a target receptor. For example, the selection of the MHC-I receptor further indicates the H-2-db allele binding site of the MHC-I receptor.

At step/operation 404, a plurality of pre-existing peptides from a peptide dataset are encoded. In particular, the plurality of pre-existing peptides are vectorized and encoded according to a selected encoding scheme. Thus, step/operation 404 may comprise selecting, or receive a selection of, a particular encoding scheme. FIG. 4B provides a diagram illustrating various steps/operations of process 400, and as shown in FIG. 4B, a peptide dataset for a plurality of pre-existing peptides 412 is received, retrieved, accessed, and/or the like from a database 130. In one example embodiment, one or more peptide datasets, including the Immune Epitope Database (IEDB), are retrieved. FIG. 4B then illustrates the pre-existing peptides 412 being vectorized and encoded such that the pre-existing peptides 412 are interpretable (and features can be extracted) by the GAN machine learning model 302. That is, at step/operation 404, the plurality of pre-existing peptides 412 are vectorized and encoded to be provided to the GAN machine learning model 302. In various embodiments, an encoding of a peptide is a data object generated for and/or output by the GAN machine learning model 302, and in some embodiments, the encoding of a pre-existing peptide 412 is generated based at least in part on user input. Generally, encoding of the plurality of pre-existing peptides at step/operation 404 may comprise generation of encoded data objects each corresponding to a pre-existing peptide.

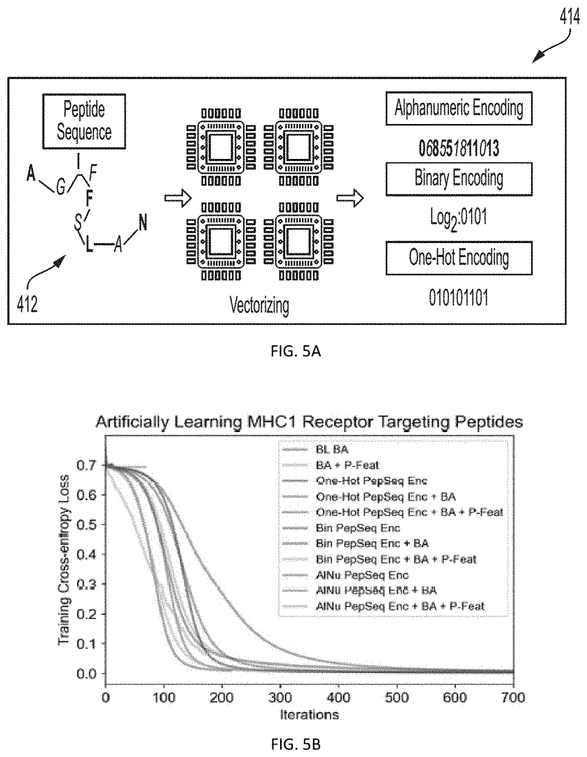

FIG. 5A illustrates example encoding schemes for encoding a pre-existing peptide 412. It will be understood that consistency is ensured by the GAN machine learning model 302 outputting a designed peptide according to the same encoding scheme of the pre-existing peptides 412 provided as input. In any regard, the pre-existing peptide 412 is vectorized according to the amino acid sequence of the peptide. Particular values may be assigned to different amino acids, and thus, a resulting vector may include values that correspond with amino acids of the pre-existing peptide 412. In one example embodiment, binary encoding is used, in which the resulting vector with amino acid-corresponding values at various indices corresponding to positions along the peptide is converted to a binary value (e.g., 0101). In another example embodiment, the pre-existing peptide 412 is encoded using one-hot encoding, in which a matrix with the length of the pre-existing peptide 412 being one dimension and the number of amino acids being another dimension. The matrix is then composed of zeros and ones, and ones demarcate a particular amino acid being present at a particular position along the length of the pre-existing peptide 412. It may be appreciated however that encoding using binary encoding and/or one-hot encoding may result in loss of structural information and density information. In another example embodiment, alphanumeric encoding may be used, in which different values corresponding to the identifying letters of the amino acids are encoded. Altogether, various ordinal encoding schemes that preserve positional information may be used for encoding the pre-existing peptide 412.

At step/operation 406, the discriminator of the GAN machine learning model 302 is configured (e.g., trained) based at least in part on selecting a plurality of physiochemical parameters for classification of binding ability of a peptide with the target receptor (e.g., received/selected in step/operation 402). While the encoding schemes discussed for step/operation 404 retain positional information of a peptide, various physiochemical properties of the peptide may not be explicitly encoded. Thus, various physiochemical parameters may be encoded and may supplement the positional encoding of a pre-existing peptide 412. In various embodiments, the physiochemical parameters that may be selected include at least one of solvation energy, binding affinity, radius of gyration, molecular force constant, unit polarization, total mass, motif, or residue position.

Thus, supplemented encodings (e.g., with positional information and physiochemical parameters) of the plurality of pre-existing peptides 412 are provided to the discriminator of the GAN machine learning model 302 in order to configure and train the discriminator to classify a pre-existing peptide 412 as binding with the target receptor or non-binding with the target receptor. The object of training the discriminator is to maximize the classification accuracy (e.g., minimize cross-entropy loss) such that designed peptides output by the generator of the GAN machine learning model 302 can be accurately classified as binding or non-binding with the target receptor.

FIG. 5B illustrates example data comparing different encoding schemes with respect to the classification ability of the discriminator of the GAN machine learning model 302. As peptide information is encoded differently across encoding schemes, each encoding scheme may result in more or less accurate classifications compared to other encoding schemes. FIG. 5B specifically illustrates iterative cross-entropy loss of the discriminator for different encoding schemes including a baseline encoding scheme, one-hot encoding scheme, binary encoding scheme, and alphanumeric encoding scheme. FIG. 5B further illustrates iterative cross-entropy loss of the discriminator for the same encoding schemes supplemented with selected physiochemical features (e.g., “Alphanumeric+P-Feat”). As illustrated, the discriminator achieves accurate classification of peptide binding ability for peptides encoding using the “Alphanumeric+P-Feat” encoding scheme in less iterations compared to other encoding schemes. That is, the discriminator converges faster for the “Alphanumeric+P-Feat” encoding scheme.

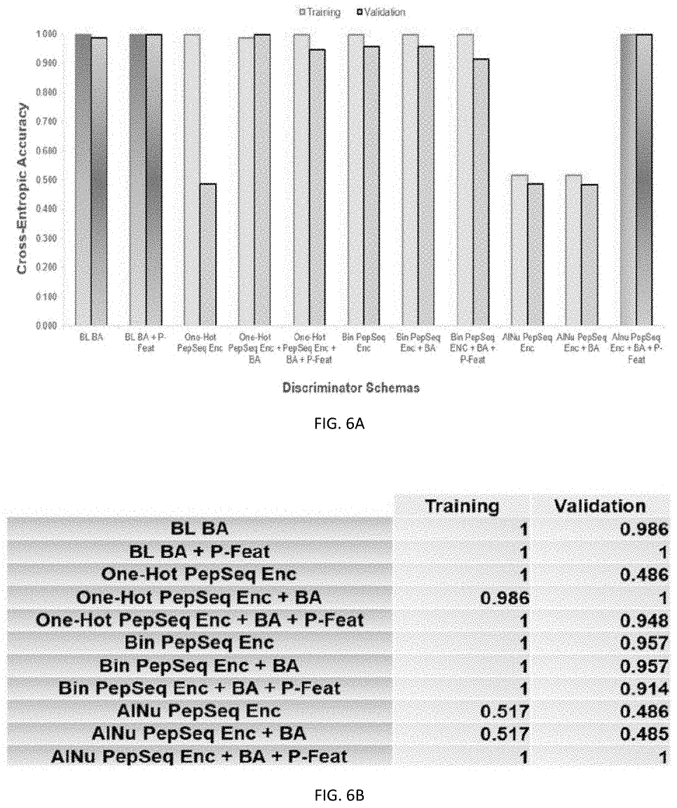

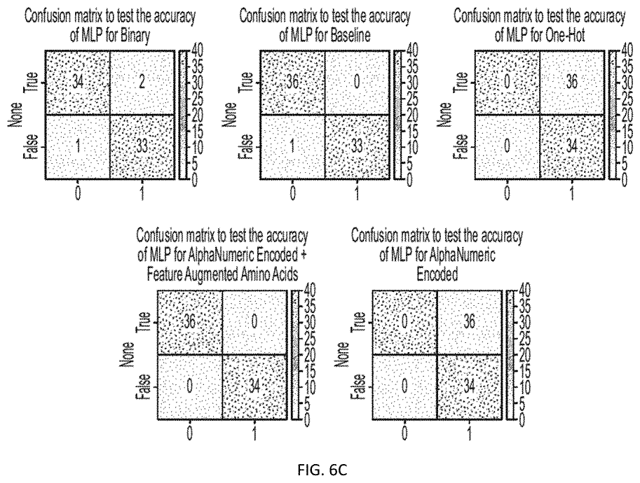

FIGS. 6A-C provide additional example data comparing classification accuracy of the discriminator for different encoding schemes. In FIG. 6A, a barplot is provided that illustrates 100% validation accuracy in the “Alphanumeric+P-Feat” encoding scheme. As shown, the addition of selected physiochemical features significantly improves both training and validation accuracy for the alphanumeric encoding scheme. FIG. 6B provides a table of values of the barplot confirming that the “Alphanumeric+P-Feat” encoding scheme has 100% accuracy in both training and validation for discriminator classification, thereby suggesting that the “Alphanumeric+P-Feat” encoding scheme may be ideal for encoding pre-existing peptides 412, at least for classification by the discriminator. FIG. 6C then illustrates confusion matrices for different encoding schemes, further indicating the classification accuracy of the discriminator for the encoding schemes describes in FIGS. 6A-B. As again shown, the “Alphanumeric+P-Feat” encoding scheme has 100% validation accuracy, with 36 true positive samples and 34 true negative samples.

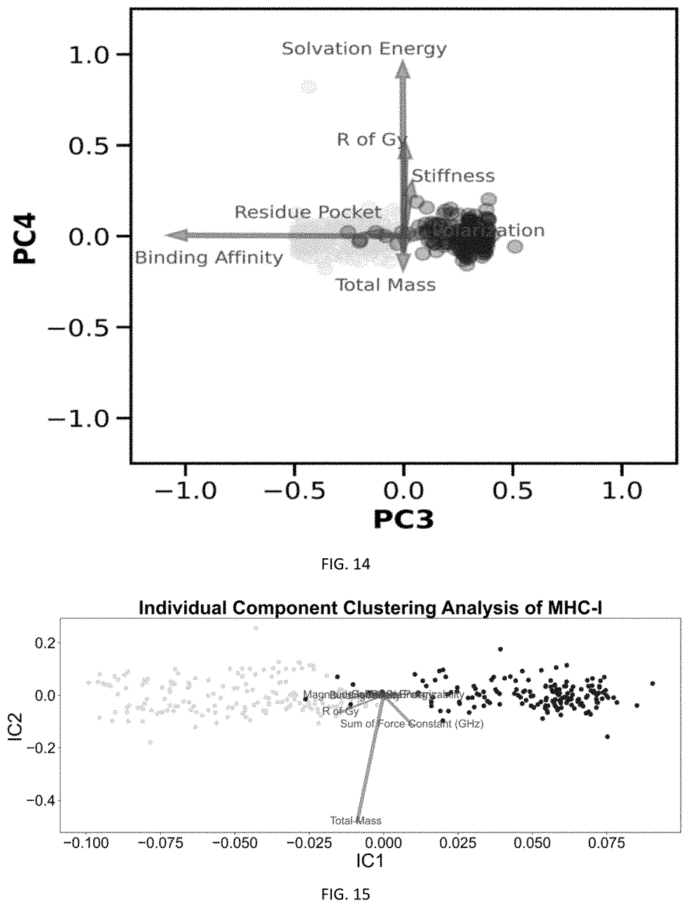

FIG. 14 illustrates example data assessing the encoding mechanism relative to all the computational techniques, according to one embodiment. Specifically, FIG. 14 illustrates an exemplary method, in which SNN_F is through alphanumeric encoding, retains all relevant information about the antigen as illustrated in the receiver operating curve and visualized AUC.

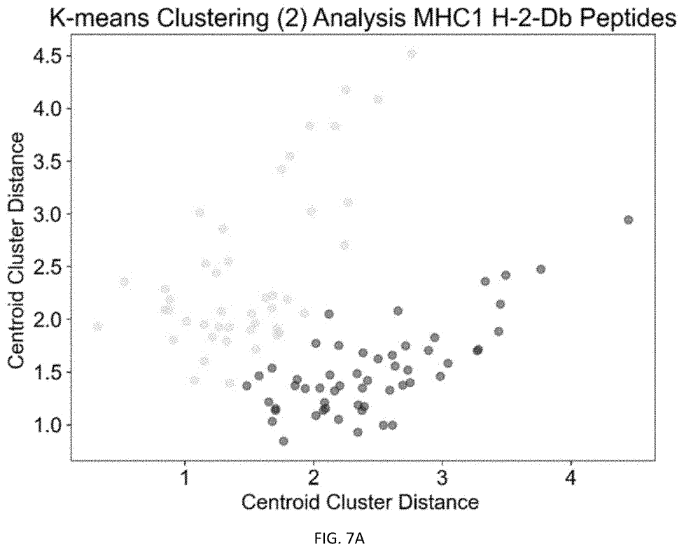

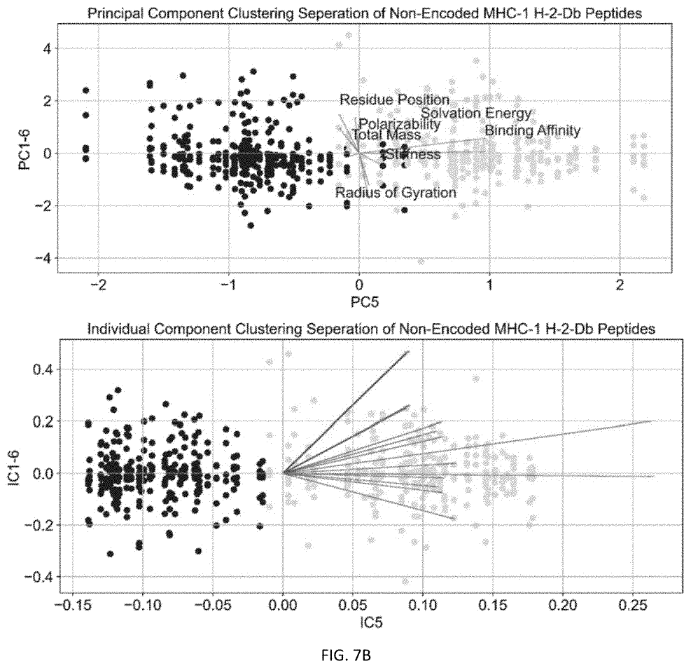

In various embodiments, the physiochemical parameters used to supplement encoding of the pre-existing peptides 412 and to use for classification of binary binding ability of a pre-existing peptide 412 are selected to maximize the classification accuracy of the discriminator. FIGS. 7A-B illustrate example clustered data showing the effective selection of features for distinguishing between binding peptides and non-binding peptides. In FIG. 7A, a k-means clustering (e.g., k=2) is performed to cluster binding peptides and non-binding peptides with respect to selected physiochemical parameters, and as illustrated, binding peptides and non-binding peptides are significantly distinguishable using the selected physiochemical parameters. FIG. 7B illustrates example data according to both principal component clustering and individual component clustering using selected physiochemical parameters of residue position, solvation energy, binding affinity, polarizability, total mass, stiffness, and radius of gyration. As also illustrated in FIG. 7B, binding peptides and non-binding peptides are well-distinguishable and well-discriminated. In various embodiments, particular physiochemical parameters are selected using clustering techniques that maximize distance in a physiochemical parameter feature space between a binding peptide cluster and a non-binding peptide cluster. In various embodiments, physiochemical parameters for a peptide are determined based at least in part on geometrically optimizing a structure of the peptide (e.g., using simulations, such as ORCA quantum chemistry package).

FIG. 14 illustrates an example principal component analysis on how artificial intelligence really understands the molecular factors of antigen presentation by MHC-I, according to one embodiment. The ideal outcome would be a complete splitting of the dataset into two groups representing strong binders and weak binders, respectively.

FIG. 15 illustrates an example individual component analysis on how artificial intelligence understands the molecular factors of antigen presentation by MHC-I, according to one embodiment. The ideal outcome would be a complete splitting of the dataset into two groups representing strong binders and weak binders, respectively. The individual component analysis is a second or higher order resolving technique, which demonstrates GAN's deep intrinsic understanding of antigen presentation.

Returning to FIG. 4A, an aptameric peptide library is then generated. The aptameric peptide library comprises a plurality of designed peptides output by the generator of the GAN machine learning model 302 using the plurality of pre-existing peptides. As understood by those of skill in the field to which the present disclosure pertains, the GAN machine learning model 302 operates iteratively by maximizing designed peptide output by the generator while minimizing classification ability of the discriminator, essentially fooling the discriminator. The generator is configured to generate designed peptides similar to and/or based at least in part on the plurality of pre-existing peptides, and both designed peptides and pre-existing peptides are provided to the discriminator for classification. The binary cross-entropy loss of the discriminator is then provided to the generator to update generation of designed peptides, until a binary cross-entropy loss threshold of the discriminator is satisfied. Alternatively, the iterative generation and classification by both the generator and the discriminator is performed until a threshold number of iterations is reached. In any regard, the generator generates feasible and functional designed peptides that bind to the target receptor. Thus, as illustrated in FIG. 4B, the GAN machine learning model 302 outputs designed peptides 304 after iterative generation and refining according to the classification ability and accuracy of the discriminator, and the designed peptides 304 are capable of binding with the target receptor. FIG. 4B further illustrates synthesis of the designed peptides 304 and implantation of the designed peptides 304 with exosomes 418 to form augmented exosomes 420 that can target the target receptor.

In various embodiments, the generator of the GAN machine learning model 302 generates designed peptides 304 (e.g., to then be classified by the discriminator) from pre-existing peptides 412, or parent peptides, according to a configurable learning rate and various other parameters. In various embodiments, the generator uses a modified Adam algorithm to converge generation of designed peptides 304. Algorithm 1 below provides an example algorithm used by the generator to iteratively output designed peptides 304 from pre-existing peptides 412 and previously generated designed peptides 304.

Algorithm 1

| | |
|---|---|
| 1. | Set the learning rate as LR |
| 2. | Set the hyperparameter as ε |
| 3. | The learning rate function: |
| | $a . LR = \vert \sqrt[- 9]{COS ( 16 π \cdot τ )} \vert where τ are the epochs$ |
| 4. | The new Adam update step: |
| | $a . {Δτ}_{update} = \vert \sqrt[- 9]{COS ( 16 π \cdot τ )} \vert \cdot \frac{\hat{{m}_{τ}}}{\sqrt{\hat{{v}_{τ}}}}$ |
| | $\begin{matrix}b . bounded by \frac{\vert \sqrt[- 9]{COS ( 16 π \cdot τ )} \vert \cdot ( 1 - {β}_{2} )}{\sqrt{1 - {β}_{2}}} \leq \vert Δτ \vert \leq \\ \vert \sqrt[- 9]{COS ( 16 π \cdot τ )} \vert\end{matrix}$ |
| 5. | If the LR < 0.01 |
| | a. Set LR as ε · LR |
| 6. | If the LR > 0.01 |
| | a. Set LR as 1 · 10⁻⁵ |

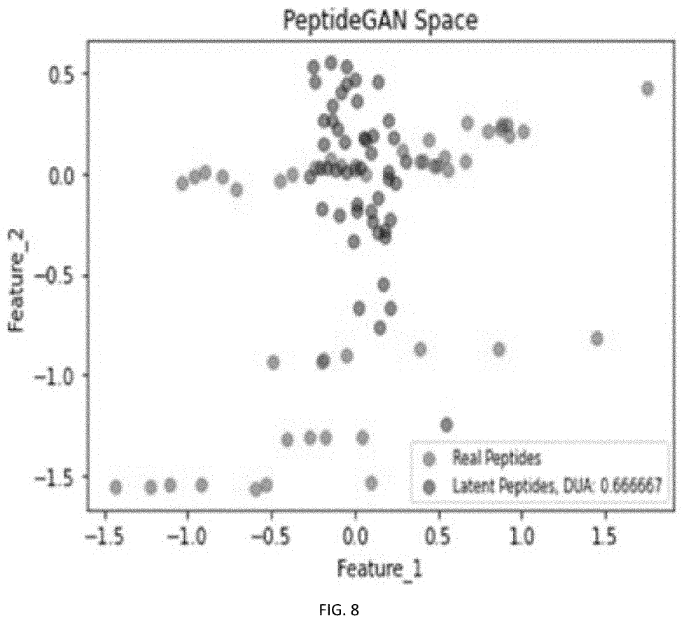

In various embodiments, generation of designed peptides 304 by the generator of the GAN machine learning model 302 can be restricted according to the selected physiochemical parameters. FIG. 8 illustrates the selection of a proper generation domain or a generative space. Specifically, in FIG. 8 a generation domain is selected to restrict Feature 1 to between −0.5 and 0.5 (e.g., Feature 1 may be one physiochemical parameter or a combination of physiochemical parameters). Restriction and selection of the generation domain enables designed peptides to be feasible, true, and functional, as generation of infeasible designed peptide with physiochemical properties outlying the selected generation domain is minimized.

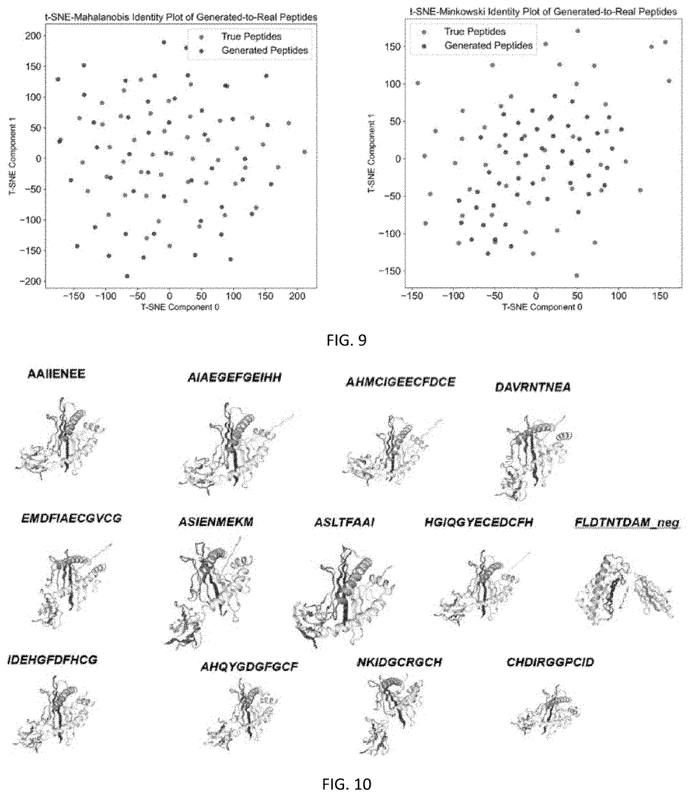

Throughput iterative generation of designed peptides 304, the generator of the GAN machine learning model 302 is configured to evaluate and remove some designed peptides 304 based at least in part on a threshold. The designed peptides 304 that are removed represent peptides that are erroneously guessed, and in some embodiments, too infeasible or non-functional. Removal of such designed peptides 304 prevents use of such designed peptides 304 as parents and prevents generation of further erroneous and infeasible designed peptides 304. FIG. 9 illustrates identity plots of parent peptides (e.g., pre-existing peptides 412, previously generated designed peptides 304) and newly-generated designed peptides 304 using t-distributed Stochastic Nearest Neighbor techniques. As shown, some newly-generated designed peptides 304 are converged or centered towards a parent peptide. Thus, in some embodiment, such converged or centered designed peptides 304 are selected for continued iterative generation and eventually for generation of the aptameric peptide library. This convergence or centering is determined according to a residual threshold, and newly-generated designed peptides 304 that do not satisfy the residual threshold are not selected for the aptameric peptide library.

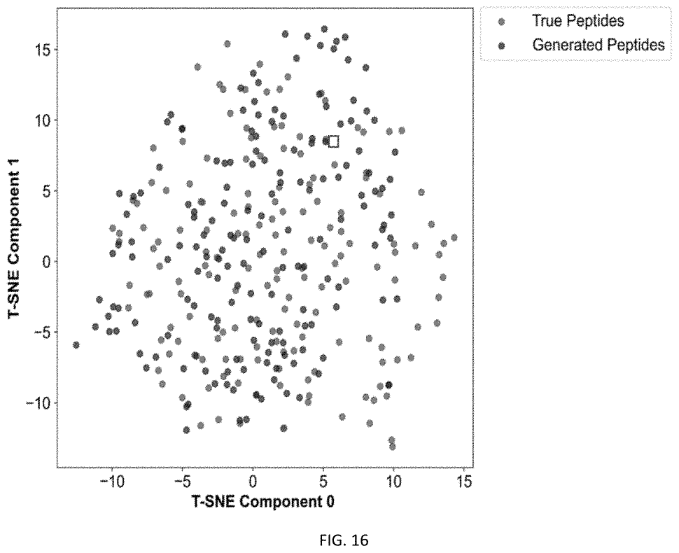

FIG. 16 illustrates an example t-distributed stochastic neighbor embedding visualization plot (t-SNE), according to one embodiment. T-SNE's main purpose is to correct all the confounding factors within the molecular dataset, specifically to scour potentially different classes of antigens. The technique may help overlay both true and generated peptides and the goal is to generate molecularly similar peptides. Selection of closest neighbors may be subjected to quality control by molecular docking. An exemplary boxed selection is shown in FIG. 16.

The table depicted in FIG. 27 illustrates example and selected designed peptides 304 associated with a parent pre-existing peptide 412. At the top of the table, designed peptides 304 that are converged to their parent pre-existing peptide 412 are listed, while designed peptides 304 that failed to converge to their parent pre-existing peptide 412 are listed at the bottom of the table. As previously discussed, convergence to a parent peptide may be determined using a residual threshold. Each designed peptide 304 and each parent pre-existing peptide 412 is listed with a quantitative binding measure (e.g., HDOCK RM SD) that may be a simulated value describing a binding ability or affinity with the target receptor, with more negative values indicating better binding with the target receptor. In various embodiments, designed peptides 304 are selected for the aptameric peptide library according to parent peptide convergence and/or quantitative binding measures. FIG. 10 illustrates visualizations of structures of various designed peptides 304 and pre-existing peptides 412 listed in the table of FIG. 27.

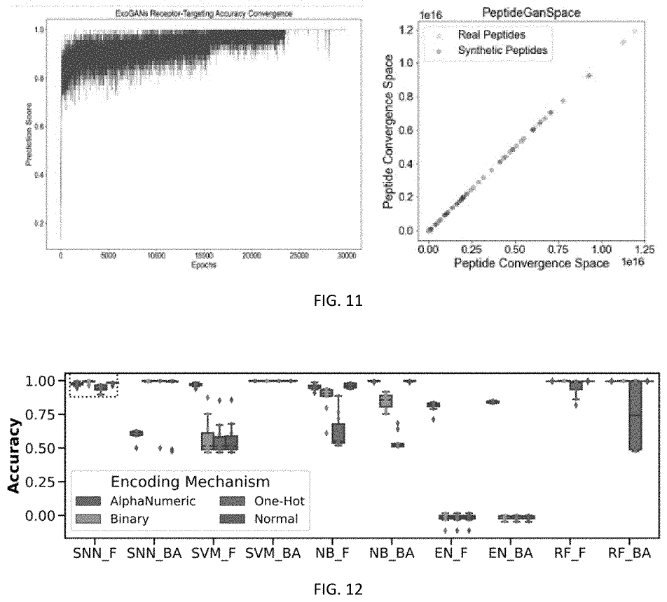

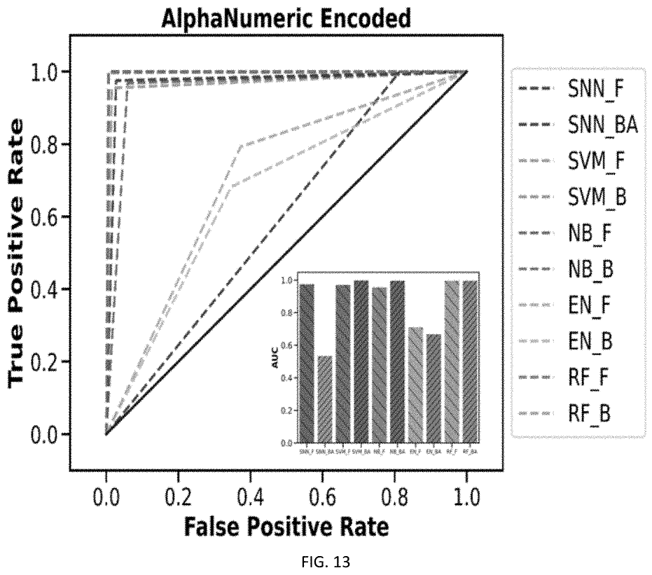

Referring now to FIG. 11, example performance plots of the GAN machine learning model 302 are provided. The first performance plot illustrates convergence of designed peptide 304 binding accuracy to the target receptor over several iterations and epochs. Specifically, the binding accuracy of the designed peptides 304 output by the GAN machine learning model 302 with the target receptor converges to 1 after some number of iterations and epochs. Meanwhile, the second performance plots generally illustrates the similarities between the designed peptides 304 output by the GAN machine learning model 302 and the pre-existing peptides 412 used as parent peptides during generation of designed peptides 304. As illustrated, the designed peptides 304 output after convergence of the GAN machine learning model 302 (e.g., as illustrated in the first performance plot) overlap the pre-existing peptides 412, indicating convergence and centering of the designed peptides 304 to parent peptides in the generative feature space.

Thus, as described, an aptameric peptide library comprising designed peptides 304 output by the GAN machine learning model 302 and satisfying various conditions such as binding ability can be generated. As previously discussed, various actions can then be performed with the aptameric peptide library. In some example instances, the aptameric peptide library is synthesized and validated using cell assays for binding with the target receptor. In one example embodiment, validation is performed using an ELISA cell assay. In various example instances, the aptameric peptide library is synthesized and implanted onto exosomes 418 to form augmented exosomes 306 for tissue targeting, drug delivery, signaling, and/or the like.

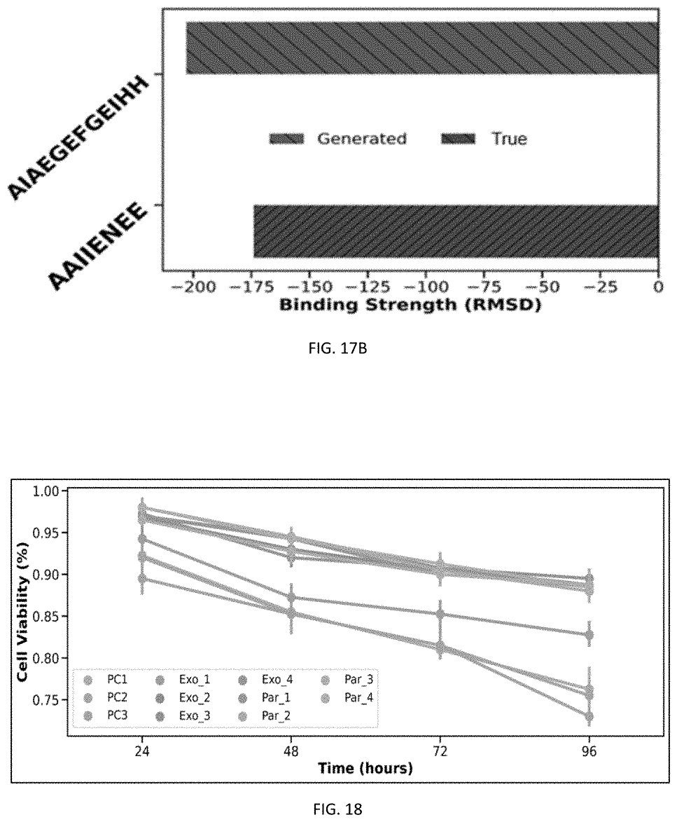

FIG. 18 illustrates example data describing resulting cell viability assay by trypan blue staining determining how cells react to stimulation, according to one embodiment. As shown in FIG. 18, the generated peptide (Exo_1) has the highest stimulation and the lowest cell viability when compared to other peptides.

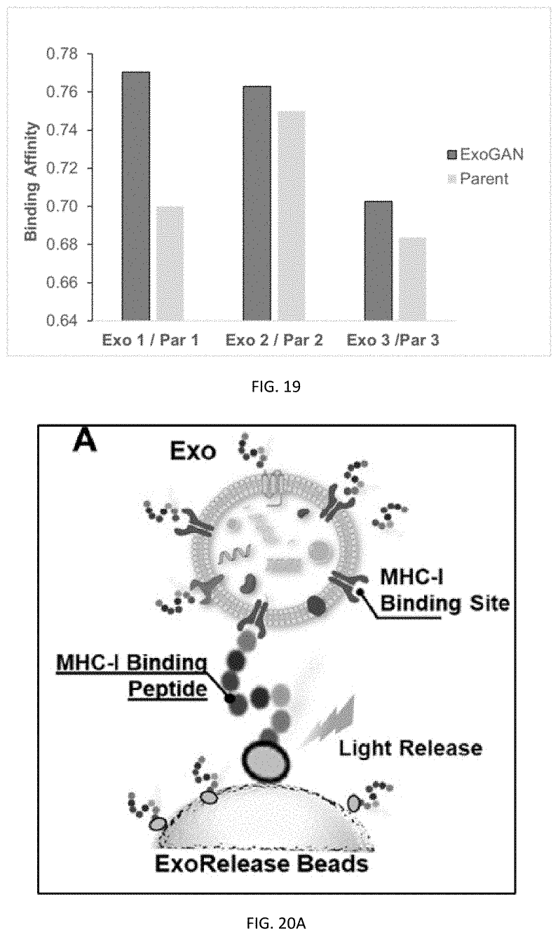

FIG. 19 illustrates example data describing the experimental binding affinity evaluation by biolayer interferometry for three pairs of peptides from earlier, according to one embodiment. As shown in FIG. 19, all the generated peptides outdo their parent peptides in terms of binding to MHC-I.

FIG. 20A is an exemplary diagram illustrating a preliminary translation of exosome vaccines by the established method using generated peptides, in accordance with an example embodiment.

FIG. 20B illustrates example data describing the concentration of peptide with respect to the size of the peptide, according to one embodiment.

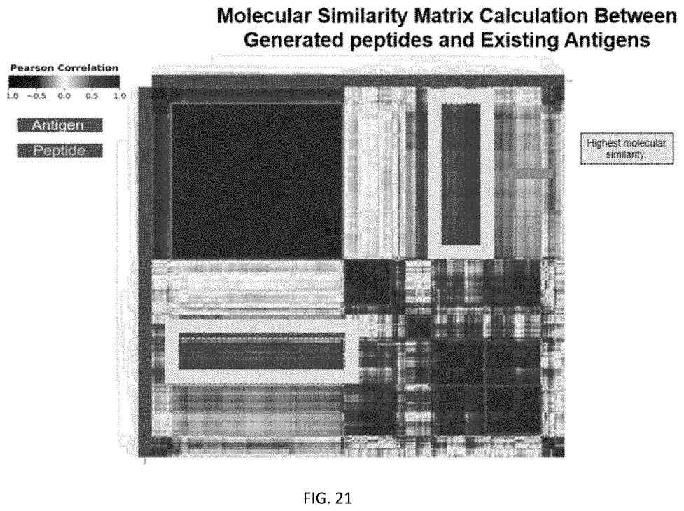

FIG. 21 illustrates example computational quality control to evaluate how the GAN generated synthetics are molecularly similar to the naturally existing cancer antigens, according to one embodiment. As shown in FIG. 21, red color indicates strong similarity, blue color indicates negative similarity.

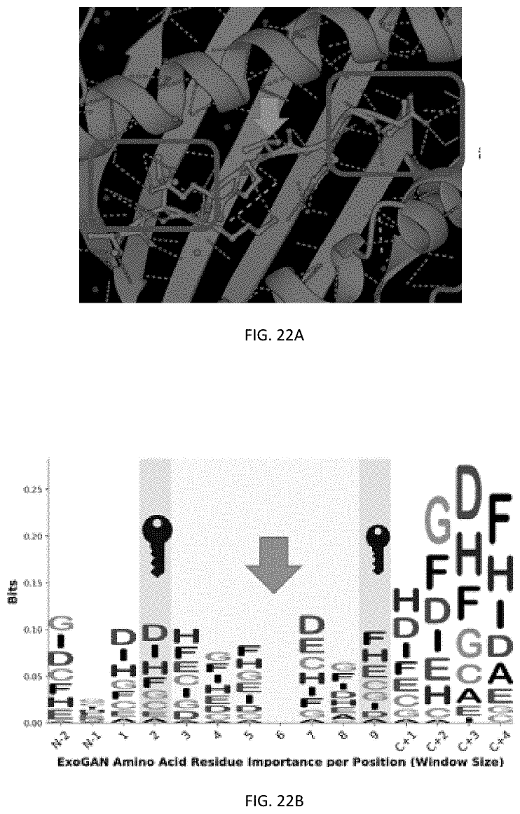

FIG. 22A illustrates the generation of synthetics in boxed regions, according to one embodiment.

FIG. 22B illustrates example data evaluating the importance of residues and their positions within MHC-I, according to one embodiment. As shown in FIG. 22B, the model may constrain the generation of synthetics to retain positional importance, regardless of residue.

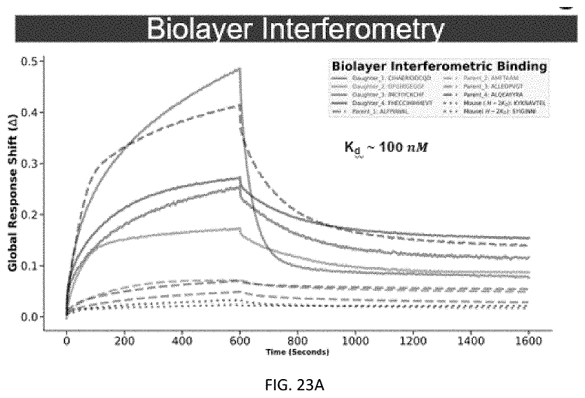

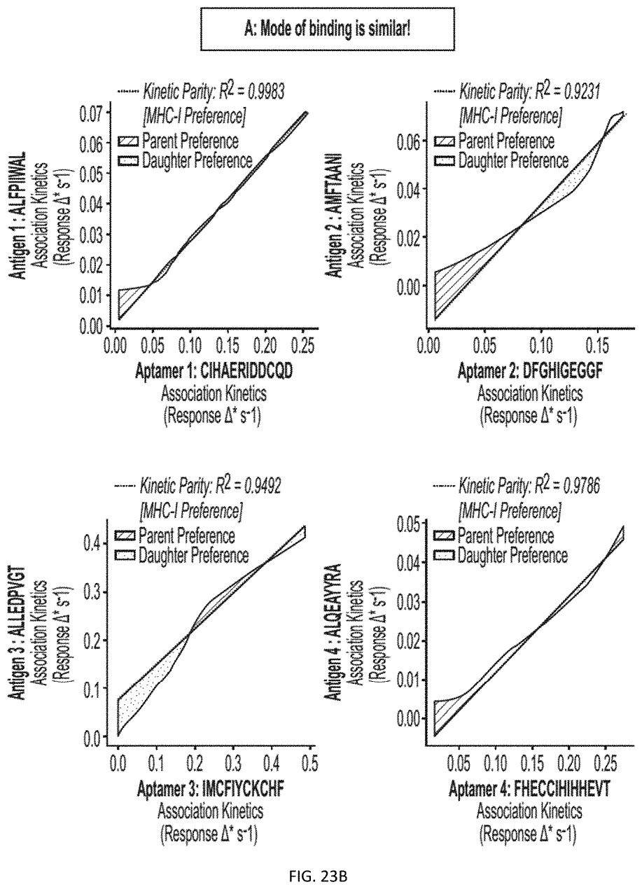

FIG. 23A illustrates example data describing experimental validation presumed biolayer interferometric binding strength, according to one embodiment.

FIG. 23B illustrates example data describing experimental validation presumed kinetics for generated synthetic (daughter) and their antigen counterpart (parent), according to one embodiment. More specifically, the parent and daughter are kinetically identical and have different MHC-I binding strength.

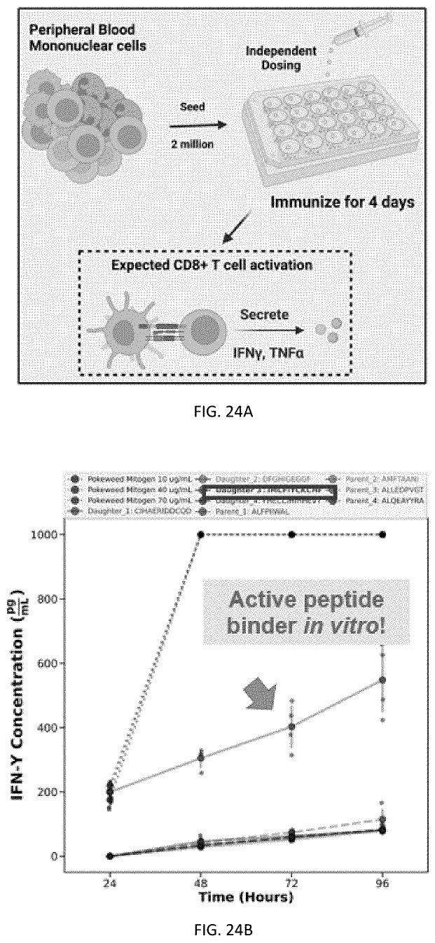

FIG. 24A illustrates an exemplary methodology workflow for an experimental validation of immunogenic activity, in accordance with various embodiments of the present disclosure.

FIG. 24B illustrates example data describing a GAN generated peptide produced immunogenic activity in four independent experiments, according to one embodiment.

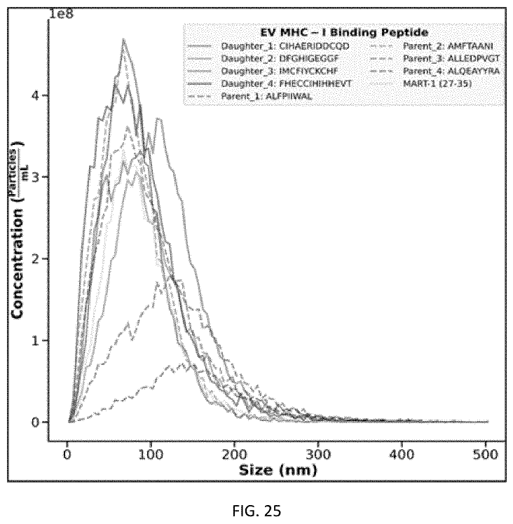

FIG. 25 illustrates example data describing size distributions detailing MHC-I+ enriched EVs developed from an end-to-end method, according to one embodiment.

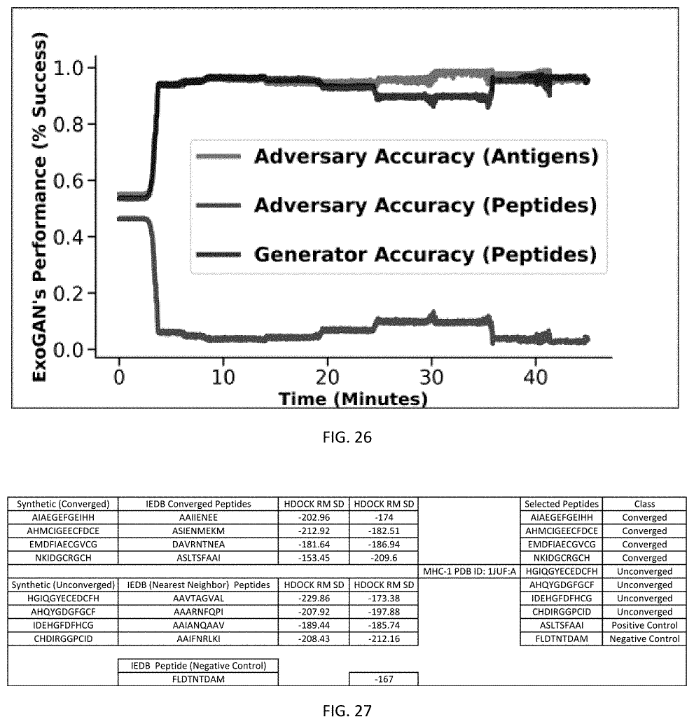

FIG. 26 illustrates example data evaluating the performance of GAN's education and manufacturing of new MHC-I binding peptides, according to one embodiment.

As such, various embodiments describe efficient, time-conserving, resource-conserving and scalable design of aptameric peptides and/or aptameric peptide libraries using GAN machine learning models and likewise solve various technical challenges related to efficiency of epitope-receptor selection. Various embodiments involve intelligent and computational-based design of aptameric peptides, or epitopes, which can form aptameric peptide libraries within a short timeframe and can further be synthesized for drug delivery and therapeutic applications. Computational-based design of aptameric peptides is more precise in targeting selected tissues and/or receptors and advantageously conserves real-world resources in designing aptameric peptides for a specific receptor. Further still, various embodiments for intelligently designing aptameric peptides are advantageously scalable and reduce variation risk across different targeting selections, thereby enabling integration in industrial and manufacturing pipelines. FIG. 29A illustrates an exemplary methodology workflow for an end-to-end pipeline to direct and molecular engineer therapeutic EVs, in accordance with various embodiments of the present disclosure.

### IV. Exemplary Computer Program Product, Methods, and Computing Entities

Embodiments of the present disclosure may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described herein with reference to block diagrams and/or flowchart illustrations. Thus, it should be understood that each block of the block diagrams and/or flowchart illustrations may be implemented in the form of a computer program product, an entirely hardware embodiment, a combination of hardware and computer program products, and/or apparatus, systems, computing devices, computing entities, and/or the like, carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### V. CONCLUSION

It should be understood that the examples and embodiments described herein are for illustrative purposes only and that various modifications or changes in light thereof will be suggested to persons skilled in the art and are to be included within the spirit and purview of this application. Although the present disclosure is considered complete and comprehensive, additional context and insight may be gleaned from the appendices attached alongside this specification (which describe generally systems, apparatuses, and methods in accordance with embodiments herein). It should be understood that the examples and embodiments in Appendices A-Care also for illustrative purposes and are non-limiting in nature. The contents of Appendices A-C are incorporated herein by reference in their entirety.

Many modifications and other embodiments of the present disclosure set forth herein will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

1. A method comprising:
receiving, by one or more processors, a selection of a target receptor;
encoding, by the one or more processors, a plurality of pre-existing peptides from a peptide dataset;
configuring, by the one or more processors, a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and
generating, by the one or more processors, an aptameric peptide library comprising a plurality of designed peptides output by the generator of the GAN machine learning model, the aptameric peptide library being specific to the target receptor.

2. The method of claim 1, wherein the plurality of designed peptides are output based at least in part on an iterative minimization of a classification accuracy of the discriminator.

3. The method of claim 2, wherein an iteration-specific classification accuracy of the discriminator is provided to the generator such that the generator outputs a designed peptide based at least in part on a previously-output designed peptide and the iteration-specific classification accuracy of the discriminator.

4. The method of claim 2, wherein the classification accuracy of the discriminator is determined based at least in part on a binary cross-entropy loss of the discriminator.

5. The method of claim 2, wherein the plurality of physiochemical parameters are selected based at least in part on a maximation of the classification accuracy of the discriminator.

6. The method of claim 1, wherein the plurality of physiochemical parameters comprise at least one of solvation energy, binding affinity, radius of gyration, molecular force constant, unit polarization, total mass, motif, or residue position.

7. The method of claim 1, further comprising synthesizing at least one designed peptide of the aptameric peptide library.

8. The method of claim 1, further comprising experimentally validating that the plurality of designed peptides of the aptameric peptide library binds to the target receptor using an enzyme-linked immunosorbent assay.

9. The method of claim 1, wherein the plurality of pre-existing peptides are encoded according to one of an alphanumeric encoding scheme, a binary encoding scheme, or a one-hot encoding scheme.

10. The method of claim 1, wherein the plurality of designed peptides satisfy a similarity threshold with the plurality of pre-existing peptides.

11. The method of claim 1, wherein the discriminator comprises at least one of a random forest classifier, a clustering classifier, or a multilayer perceptron.

12. The method of claim 1, further comprising attaching one or more of the designed peptides of the aptameric peptide library onto a surface of an exosome or extracellular vesicle (EV) to form an augmented exosome or augmented EV, wherein one or more designed peptides target the augmented exosome or augmented EV to a target cell presenting the target receptor.

13. An apparatus comprising at least one processor and at least one memory including program code, the at least one memory and the program code configured to, with the processor, cause the apparatus to at least:
receive a selection of a target receptor;
encode a plurality of pre-existing peptides from a peptide dataset;
configure a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and
generate an aptameric peptide library comprising a plurality of designed peptides output by the generator of the GAN machine learning model, the aptameric peptide library being specific to the target receptor.

14. The apparatus of claim 13, wherein the plurality of pre-existing peptides are encoded according to one of an alphanumeric encoding scheme, a binary encoding scheme, or a one-hot encoding scheme.

15. The apparatus of claim 13, wherein the plurality of designed peptides satisfy a similarity threshold with the plurality of pre-existing peptides.

16. The apparatus of claim 13, wherein the discriminator comprises at least one of a random forest classifier, a clustering classifier, or a multilayer perceptron.

17. A computer program product comprising at least one non-transitory computer-readable storage medium having computer-readable program code portions stored therein, the computer-readable program code portions comprising:
an executable portion configured to
receive a selection of a target receptor;
an executable portion configured to encode a plurality of pre-existing peptides from a peptide dataset;
an executable portion configured to configure a generative adversarial network (GAN) machine learning model comprising a generator and a discriminator, the generator configured to output a designed peptide similar to the plurality of pre-existing peptides and the discriminator configured to classify a binding ability of the designed peptide with the target receptor according to a plurality of physiochemical parameters; and
an executable portion configured to generate an aptameric peptide library comprising a plurality of designed peptides output by the generator of the GAN machine learning model, the aptameric peptide library being specific to the target receptor.

18. The computer program product of claim 17, wherein the plurality of pre-existing peptides are encoded according to one of an alphanumeric encoding scheme, a binary encoding scheme, or a one-hot encoding scheme.

19. The computer program product of claim 17, wherein the plurality of designed peptides satisfy a similarity threshold with the plurality of pre-existing peptides.

20. The computer program product of claim 17, wherein the discriminator comprises at least one of a random forest classifier, a clustering classifier, or a multilayer perceptron.

21. A method for forming a targeted therapeutic exosome or EV comprising a designed peptide having affinity for a cellular receptor comprising: selecting one or more designed peptides from the aptameric peptide library of claim 1 and attaching the one or more designed peptides to a surface of an exosome or EV thereby forming the targeted therapeutic exosome or EV; wherein the cellular receptor is the selected target receptor and wherein the exosome or EV comprises or contains a drug or therapeutic.

22. A method of delivering a drug or therapeutic to a target cell or tissue comprising contacting the cell or tissue with the therapeutic exosome or EV of claim 21, wherein the cell or tissue presents the target receptor.
